(19) United States
(12) Patent Application Publication
(10) Pub. No.: US 2004/0132970 A1
(43) Pub. Date: Jul. 8, 2004

(54) Dermaseptin-derived peptides and their use in delivery systems

(75) Inventors: Abraham Loyter, Jerusalem (IL); Haim Gilon, Jerusalem (IL); Adolf Graessman, Berlin (DE); Amram Mor, Haifa (IL); Ilana Hariton-Gazal, Rehovot (IL)

Correspondence Address: MERCHANT & GOULD PC, P.O. BOX 2903, MINNEAPOLIS, MN, 55402-0903, US

(73) Assignee: YISSUM RESEARCH DEVELOPMENT COMPANY OF THE HEBREW UNIVERSITY OF JERUSALEM, Jerusalem (IL)

(21) Appl. No.: 10/455,698

(22) Filed: Jun. 5, 2003

(30) Foreign Application Priority Data
Jun. 6, 2002 (IL) 150087

(51) Int. Cl. C07K 14/47

(52) U.S. Cl. 530/350; 514/12

(57) ABSTRACT

The present invention is concerned with new delivery systems for intracellular and intranuclear import of desired constituents based on dermaseptin-derived peptides. For that purpose, the present invention provides a chimeric molecule, comprising a dermaseptin-derived peptide and at least one biologically or pharmaceutically active constituent. The chimeric molecule of the invention may further comprise a peptide having nuclear localization signal (NLS)-like properties. In addition, a fusion peptide is provided, consisting of a dermaseptin-derived peptide and a peptide with NLS-like properties. The present invention also provides compositions and systems for the intracellular delivery of active constituents, and methods for screening cell-permeable nuclear import inhibitors and for detecting changes in intracellular levels of proteins and nucleic acids.

### FIELD OF THE INVENTION

The present invention relates generally to intracellular and intranuclear import. In particular, the present invention is concerned with providing new delivery systems for intracellular and intranuclear import of desired constituents based on dermaseptin-derived peptides, which are significantly more efficient than what is currently available in the art.

### BACKGROUND OF THE INVENTION

All publications mentioned throughout this application are fully incorporated herein by reference, including all references cited therein.

Introduction of macromolecules such as proteins [Rojas, M. et al. (1998) Nat. Biotech. 16, 370-375] or nucleic acids (DNA or RNA) [Prochiantz, A. (1996) Curr. Opin. Neurobiol. 6, 629-634] into living cells by direct needle microinjection [Capecchi, M. R. (1980) Cell 22, 479-488] or electroporation [Neumann, E. (1982) EMBO 7, 841-845] has been used as a tool to study various aspects of intracellular processes. Evidently, these approaches are limited to in vitro systems, namely cultured mammalian cells. However for clinical use, especially in the fields of drug delivery and gene therapy, methods that allow the release of macromolecules into the intracellular or nuclear compartments of cells in the living organism are required. Liposomes or reconstituted viral envelopes loaded with proteins or nucleic acids have been employed as carriers that allow the delivery of their content into cells of specific tissues [Felgner, P. L. (1987) Proc. Natl. Acad. Sci. USA 84, 7413-7417; Cepko, C. L. (1984) Cell 37, 1053-1062]. Nevertheless, as demonstrated by numerous studies, such vesicular carriers are taken into the cells via a process of receptor-mediated endocytosis, which results in extensive degradation of their content by endosomal or lysosomal enzymes.

Evidently this renders such loaded vesicles inefficient carriers, characterized by low yield of cargo delivery.

During the last few years it has become apparent that certain small proteins and peptides are able to directly cross cell plasma membranes without being susceptible to degradation by the intra-endosomal enzymes. A number of natural proteins and peptides of various origins, as well as synthetic peptides, have been defined as Cell Penetrating Proteins or Peptides (CPPs), due to their ability to penetrate cell plasma membranes independently of transporters or specific receptors [Lindgren, M. et al. (2000) TIPS 21, 99-103]. Mastoparan, a peptide derived from the Drosophila Antennapedia protein (penetratins) [Higashijima, T. et al. (1990) J. Biol. Chem. 265, 14176-14186], Transportan [Pooga, M. et al. (1998) FASEB 12, 67-77] and the HIV-1 Tat protein have been shown to penetrate into intact cells and even deliver protein molecules, as well as nucleic acids, that have been covalently attached to them [Vives, E. et al. (1994) J. Virol. 68, 3343-3353]. Similar to the carrier protein itself, also the conjugates penetrate into the recipient cells via a non-endocytic process, thus escaping hydrolysis by the lysosomal hydrolytic enzymes. The translocation process of the CPPs was attributed to the activity of a specific domain within the carrier proteins, which has been termed Protein Translocation Domain (PTD) [Lindgren, M. et al. (2000) id. ibid.]. The ability of the HIV-1 Tat protein, for example, to cross cell plasma is due to a cluster of positively charged amino acids containing mostly arginine residues [Vives, E. et al. (1997) J. Biol. Chem. 272, 16010-16017]. A peptide bearing this cluster—which has been defined as ARM (Arginine Rich Motif)—is able by itself to cross plasma membranes of living mammalian cells.

The biological function of the Tat ARM domain (amino acids 48-60) is to serve as a Nuclear Localization Signal (NLS), promoting nuclear import of the HIV-1 Tat protein. The intranuclear presence of the Tat protein in virus-infected cells is essential for allowing accurate transcription of the virus genome. Also, the NLS of the HIV-1 karyophilic Rev protein is characterized by a cluster of arginine residues which, as that of Tat, is called ARM.

Recently it has been demonstrated that various arginine-rich peptides—including the Rev-ARM—share the same property, namely are able to cross the cell plasma membrane [Futaki, S. et al. (2000) J. Biol. Chem. 17]. These arginine-rich peptides, similar to the ARM peptide, are able to serve as carriers and to deliver covalently attached macromolecules into intact cells. A somewhat different strategy for protein delivery has recently been described [Morris, M. C. et al. (2001) Nat. Biotechnol. 19, 1173-1176]. A short amphipatic peptide, designated Pep-1, was shown to efficiently deliver, into mammalian cells, proteins that were mixed with it without the need of covalent attachment [Morris, M. C. et al. (2001) id ibid.].

Here, the inventors describe new fusion peptides based on the peptide S4₁₃ [Mor, A., and Nicolas, P. (1994) J. Biol. Chem. 269, 1934-1939; Feder, R. et al. (2000) J. Biol. Chem. 275, 4230-4238], with cell penetrating properties, which was derived from Dermaseptin S4, from the Dermaseptin family [Nicolas, P. and Mor, A. (1995) Annu. Rev. Microbiol. 4, 277-304]. The dermaseptins are a large family of antimicrobial peptides, found on the skin of frogs from the Phylloinedusinae genus [Mor, A. et al. (1991) Biochemistry 30, 8824-8830]. These peptides were shown to be cytolytic in a broad spectrum of pathogenic microorganisms such a bacteria, protozoa, yeast and filamentous fungi [Mor et al. (1991) id ibid; Pouny, Y. et al. (1992) Biochemistry 31, 12416-12423]. Among the natural dermaseptins, dermaseptin S4 is highly toxic to erythrocytes [Feder, R. et al. (2001) Peptides 22, 1683-1690]. Although the exact mechanism of action of these antimicrobial peptides is not yet fully understood, it appears that they destabilize target cells membranes, causing cell death. The dermaseptins are linear polycationic peptides composed of 28 to 34 amino acids and possess amphipatic alpha helix in apolar solvents [Mor, A. et al. (1994a) Biochemistry 33, 6642-6650]. Using liposomes as an experimental system it has been shown that the alpha helical peptide interacts with the liposomal phospholipids [Mor et al. (1994a) id ibid; Mor, A. and Nicolas, P. (1994) id ibid; Mor, A. et al. (1994b) J. Biol. Chem. 269, 16].

Based on the inventors' herein described results, an aim of the present invention is to provide new intracellular delivery systems that overcome the disadvantages of prior art systems.

Thus, the present invention provides chimeric molecules that can be used as delivery systems for the transport of molecules from the extracellular into an intracellular compartment, and wherein said chimeric molecules comprise dermaseptin-derived fusion peptides.

Other purposes and advantages of the invention will become apparent as the description proceeds.

### SUMMARY OF THE INVENTION

In a first aspect, the present invention provides a chimeric molecule, wherein said chimeric molecule comprises a dermaseptin-derived peptide and at least one biologically or pharmaceutically active constituent. According to the invention, said dermaseptin-derived peptide preferably comprises the sequence substantially as defined in SEQ ID NO:2 or functional analogues, derivatives or fragments thereof.

The chimeric molecule may further comprise a peptide having nuclear localization signal (NLS)-like properties, wherein said dermaseptin-derived peptide, together with the peptide having NLS-like properties, preferably comprise the sequence substantially as defined in any one of SEQ ID NO:3, SEQ ID NO:4 and SEQ ID NO:6 and functional analogues, derivatives or fragments of any of said sequences.

In one embodiment, said active constituent comprised in the chimeric molecule of the invention may be selected from amino acids, oligopeptides, small peptides, polypeptides, proteins, nucleotides, oligonucleotides, nucleic acids, drugs and fluorescently, radioactively or magnetically labeled chemical moieties.

In another aspect, the invention provides a system for intracellular delivery of a biologically or pharmaceutically active constituent comprising the chimeric molecule of the invention.

Said delivery system of the invention is intended for the delivery of said active constituent from an extracellular compartment into an intracellular compartment, wherein said intracellular compartment may be non-nuclear or nuclear.

A further aspect of the invention aims to provide a pharmaceutical composition for intracellular delivery of a pharmaceutically active constituent, wherein said composition comprises as active ingredient a chimeric molecule as defined by the invention.

Thus, the pharmaceutical composition of the invention is intended for the delivery of a pharmaceutically active constituent into a non-nuclear or nuclear intracellular compartment.

The pharmaceutically active constituent to be delivered by the composition of the invention may be, but not limited to, amino acids, oligopeptides, small peptides, polypeptides, proteins, nucleotides, oligonucleotides, nucleic acids and drugs. For the delivery of said pharmaceutically active constituent into the nuclear compartment, the composition of the invention should preferably comprise a chimeric molecule comprising a peptide with NLS-like properties.

An additional aspect of the invention is the use of the chimeric molecule of the invention as a system for the delivery of an active constituent from an extracellular compartment into the intracellular compartment, wherein said intracellular compartment may be a non-nuclear or a nuclear intracellular compartment, and said active constituent is selected from the group consisting of amino acids, oligopeptides, small peptides, polypeptides, proteins, nucleotides, oligonucleotides, nucleic acids, drugs and fluorescently, radioactively or magnetically labeled chemical moieties.

The chimeric molecule to be used for the delivery of an active constituent from the extracellular into the nuclear compartment should preferably comprise a peptide having NLS-like properties.

The chimeric molecule as defined in the invention is also intended to be used in the preparation of a pharmaceutical composition for the delivery of an active constituent from an extracellular compartment into the intracellular compartment.

As will become further clear in the following Examples, the invention is especially original for providing a fusion peptide comprising a dermaseptin-derived peptide and a peptide having nuclear localization signal (NLS)-like properties.

In preferred embodiments, the fusion peptide is one of the sequences substantially as defined in SEQ ID NO:3, SEQ ID NO:4 or SEQ ID NO:6.

The fusion peptide as defined in the invention is intended to be used as a delivery system for substances from the extracellular milieu to the intracellular compartment.

Alternatively, the invention relates to the use of said fusion peptide as a delivery system for transport of substances from the extracellular milieu to the intracellular compartment.

It is also the intention of the present invention to provide a method of screening for a cell-permeable nuclear import inhibitor, wherein said method comprises the following steps:

a. providing cells, contacting said cells with a chimeric molecule as defined in the invention;

b. contacting said cells with a candidate substance;

c. detecting the import of said chimeric molecule into the nuclei of said cells;

whereby the absence of import of the chimeric molecule indicates that said candidate substance is an inhibitor of nuclear import. In this method, the chimeric molecule should preferably comprise a peptide having NLS-like properties.

Lastly, the invention provides a method for detecting changes in intracellular levels of proteins and nucleic acids, as well as of fragments thereof, comprising the following steps:

a. providing cells, contacting said cells with a chimeric molecule of the invention, wherein said chimeric molecule comprises a peptide having NLS-like properties, in which case said dermaseptin-derived peptide together with said NLS-like peptide preferably comprise the sequence substantially as defined in any one of SEQ ID NO:3, SEQ ID NO:4 or SEQ ID NO:6, as well as functional analogues, derivatives or fragments of any of said sequences, wherein at least one of said active constituents of the chimeric molecule of the invention can bind to the nucleic acid or protein whose levels are to be measured, and which chimeric molecule may optionally further comprise a second active constituent comprising a fluorescently, radioactively or magnetically labeled chemical moiety;

b. detecting the amount of said chimeric molecule in the cytoplasm and/or nuclei of said cells by suitable means; and

c. comparing the results obtained with an established control value of the non-nuclear and/or nuclear level of the nucleic acid or protein of interest, respectively;

whereby a level of the nucleic acid or protein of interest that is different from the control value of said nucleic acid or protein indicates a change on its level.

### BRIEF DESCRIPTION OF THE FIGURES

FIGS. 1a-e: Cell penetration and nuclear import of S4₁₃ and the S4₁₃-derived peptides: fluorescence microscopy observations.

FIG. 1a: HeLa cells incubated for 30 minutes with 5 μM of S4₁₃;

FIG. 1b: HeLa cells incubated for 30 minutes with 5 μM of PV-S4₁₃;

FIG. 1c: HeLa cells incubated for 30 minutes with 5 μM of rPV-S4₁₃ peptides at 37° C.;

FIG. 1d: HeLa cells incubated for 30 minutes with 5 μM of S4₁₃ at 4° C.;

FIG. 1e: HeLa cells incubated for 30 minutes with 5 μM of PV-S4₁₃ at 4° C.

Note that only in FIG. 1b the nuclei and mainly the nucleoli are highly fluorescent while in the other panels (FIGS. 1a, 1c, 1d, 1e) cytoplasmic retention is observed.

FIGS. 2a-d: Nuclear import of PV-S4₁₃ in permeabilized HeLa cells: fluorescence microscopy observations. All experimental conditions were as described in Experimental Procedures and in legend to Table 3. Digitonin permeabilized HeLa cells were incubated in the presence of reticulocyte extract at 30° C. for 1 hour with 1 μM of the following:

FIG. 2a: S4₁₃;

FIG. 2b: PV-S4₁₃;

FIG. 2c: rPV-S4₁₃;

FIG. 2d: PV-S4₁₃ incubation as in FIG. 2b, but in the absence of reticulocyte extract.

FIGS. 3a-b: Cell penetration of RR-S4₁₃ and Rev-ARM peptides. Experimental conditions of peptide synthesis, labeling and incubation with cultured intact HeLa cells were as described in Experimental Procedures. Cells were incubated for 30 minutes with:

FIG. 3a: 10 μM Rev at 37° C.;

FIG. 3b: 2 μM of RR-S4₁₃.

Note that in FIG. 3b the nuclei and cytoplasm are highly fluorescent in comparison to FIG. 3a.

FIGS. 4a-d: Nuclear import of S4₁₃ and RR-S4₁₃ in microinjected cells. HeLa cells were microinjected with the fluorescently labeled peptides S4₁₃ and RR-S4₁₃ (1 μM). The injection mixture also included fluorescently labeled BSA as an injection control (data not shown). Photos were taken 2 hours after incubation at 37° C.

FIG. 4a: S4₁₃

FIG. 4b: RR-S4₁₃

FIG. 4c: S4₁₃ (phase contrast)

FIG. 4d: RR-S4₁₃ (phase contrast)

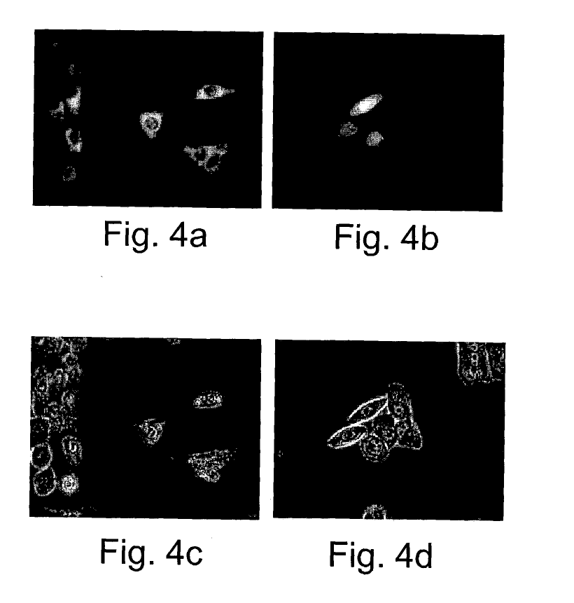

Note that RR-S4₁₃ accumulated in the nuclei (FIGS. 4b and 4d), while S4₁₃ remained in the cytoplasm (FIGS. 4a and 4c).

### DETAILED DESCRIPTION OF THE INVENTION

The following abbreviations have been used throughout this application:

ARM, Arginine Rich Motif;

BSA, Bovine Serum Albumin;

CPP, Cell Penetrating Peptide;

DCM, Dichloromethane;

DIEA, Diisopropylethylamine;

DMF, Dimethylformamide;

FCS, Fetal Calf Serum;

Fmoc, Fluorenylmethoxycarbonyl;

HBSS, Hank's Balanced Salt Solution;

HIV-1, human immunodeficiency virus type 1;

LR, Lissamine Rhodamine;

NLS, Nuclear Localization Signal;

NPC, Nuclear Pore Complex;

PBS, Phosphate Buffer Saline;

PTD, Protein Translocation Domain;

PV-S4₁₃, fusion peptide comprising S4₁₃ and the NLS sequence from SV40 T-Ag at the N-terminus;

RR-S4₁₃, fusion peptide comprising S4₁₃ and the ARM sequence of the Rev protein;

S4₁₃-PV, fusion peptide comprising S4₁₃ and the NLS sequence from SV40 T-Ag at the C-terminus

SPPS, Solid Phase Peptide Synthesis;

SV40 T-Ag, SV40 T antigen

TDW, Triple Distilled Water;

TFA, Trifluoroacetic Acid;

TOF-MS, Time of Flight Mass Spectrometry

The results herein presented by the inventors demonstrate that chimeric molecules, exemplified by the fluorescently labeled antimicrobial dermaseptin S4-derived peptide S4₁₃, readily penetrate into intact HeLa cells and accumulate within the cytoplasm of these cells. Kinetic studies revealed that penetration of the chimeric molecule was fast, occurring within 5 min of incubation at either 37° C. or 4° C. Penetration into cultured cells was neither blocked by the addition of excess unlabeled (non-fluorescent) peptides nor with incubation at 4° C., and it also occurred in ATP-depleted cells (Table 2). All these results strongly indicate that penetration of the chimeric molecule comprising dermaseptin-derived peptides was a receptor independent process and probably does not occur via the endocytic pathway. Based on these results the chimeric molecules described in the invention shall be considered as novel CPPs.

Hence, in a first aspect, the present invention provides a chimeric molecule, wherein said chimeric molecule comprises a dermaseptin-derived peptide and at least one biologically or pharmaceutically active constituent. According to the invention, said dermaseptin-derived peptide preferably comprises the sequence substantially as defined in SEQ ID NO:2 or functional analogues, deriyatives or fragments thereof.

Being relatively small (13-28 amino acid residues), the S4 and S4₁₃ dermaseptin-derived peptides would be expected to, following their translocation into the cytosol, passively diffuse into the intranuclear space [Feldherr, C. M. and Akin, D. (1994) Int. Rev. Cytol. 151, 183-228]. However, surprisingly, the inventors observed that in intact, as well as in digitonin-permeabilized HeLa cells, these peptides are non-karyophilic. This failure to diffuse into the nuclear compartment may be due to their tendency to undergo self-aggregation, which could lead to the formation of high molecular weight aggregates, themselves too large to enter the nucleus by passive diffusion. To circumvent this limitation, the inventors attached an NLS-like sequence to the peptide S4₁₃ and converted it into a karyophilic CPP, namely, a peptide that accumulated within the nuclei of the recipient cells while retaining its cell penetration properties.

Therefore, the chimeric molecule of the invention may optionally further comprise a peptide having NLS-like properties, in which case said dermaseptin-derived peptide, together with said NLS-like peptide preferably comprise the sequence substantially as defined in any one of SEQ ID NO:3, SEQ ID NO:4 or SEQ ID NO:6, as well as functional analogues, derivatives or fragments of any of said sequences.

The peptides defined by SEQ ID NO:3, SEQ ID NO:4 and SEQ ID NO:6 may also be considered as dermaseptin-derived peptides.

By functional analogues, derivatives or fragments as used herein it is meant any peptide sequence having substantially the same activity as SEQ ID NO:2, SEQ ID NO:3, SEQ ID NO:4 or SEQ ID NO:6 as described throughout this application.

The SV40 T-Ag NLS—whose nuclear import is mediated by the importin alpha-beta heterodimer—covalently attached to the S4₁₃ peptide resulted in the peptides PV-S4₁₃ (SEQ ID NO:4) and S4₁₃-PV (SEQ ID NO:3). Experiments using digitonin-permeabilized cells as an assay system have clearly demonstrated that nuclear import of both peptides is subjected to the same features that characterize active nuclear import (Example 3). Interestingly, the covalent attachment of the SV40 T-Ag NLS to S4₁₃ did not have any effect on the cell permeability properties of the three peptides PV-S4₁₃, S4₁₃-PV and rPV-S4₁₃ which, similar to S4₁₃, readily penetrated into the cultured HeLa cells.

Similar to the SV40 T-Ag NLS, the Rev ARM also conferred karyophilic properties on the S4₁₃ peptide, without affecting its cell penetration abilities. Thus, the Rev-ARM remained biologically active following its attachment to S4₁₃. The fact that the Rev-ARM peptides were biologically active within the RR-S4₁₃ can be inferred also from the experiments using the in vitro nuclear assay system (Example 4). Import of the RR-S4₁₃ into nuclei of the permeabilized HeLa cells, as opposed to nuclear import mediated by the SV40 T-Ag NLS [Efthymiadis, A. et al. (1998) J. Biol. Chem. 273, 1623-1628], did not require the addition of external cytosolic factors. On the contrary, external cytosolic factors exert inhibition of ARM-mediated nuclear import. Such inhibition appears to be due to non-specific electrostatic interactions between the positively charged Rev-ARM and negatively charged molecules present in the cytosolic extracts, as can be inferred from the inventors' recent experiments (data not shown). However, the inhibition observed by GTPγS and free Rev peptide of the Rev-ARM mediated nuclear import clearly indicates that such import is mediated by a specific cellular receptor, which is almost certainly importin beta [Truant, R. and Cullen B. R. (1999) Mol. Cell Biol. 19, 1210-1217]. Thus, the S4₁₃-derived fusion proteins may be used as efficient carriers for the delivery of a variety of molecules into intact or permeabilized cultured cells, in the presence or absence of external cytosolic factors.

The active constituent is preferably linked to the dermaseptin-derived peptide in the presence or absence of an NLS-like sequence. This linking can be achieved, for example, by a direct chemical bond or through a spacer, which could be any cleavable or non-cleavable cross-linking agent, for example. Preferably, the chemical bond is a covalent bond. The spacer may be a peptide of up to five amino acids, which provides three-dimensional flexibility to the chimeric molecule. Notably, a chimeric molecule of the invention, comprising the dermaseptin-derived peptide and an NLS sequence, the active component may be linked to any one of the moieties, i.e., it may be linked to either the dermaseptin-derived or the NLS-derived moiety.

In one embodiment of the chimeric molecule of the invention, the comprised active constituent may be any one of, but not limited to, amino acids, oligopeptides, small peptides (up to 20 amino acids), polypeptides, proteins, nucleotides, oligonucleotides, nucleic acids, and drugs and fluorescently, radioactively or magnetically labeled chemical moieties.

In another aspect, the invention provides a system for intracellular delivery of a biologically or pharmaceutically active constituent comprising the chimeric molecule of the invention.

Said delivery system of the invention is intended for the delivery of said active constituent from an extracellular compartment into an intracellular compartment, wherein said intracellular compartment may be non-nuclear or nuclear.

In the absence of an NLS-like sequence, the chimeric molecule of the invention may deliver its cargo to the cell cytoplasm, while comprising the NLS, the cargo shall be carried into the cell nucleus. The advantage of using S4₁₃ as a carrier to deliver molecules into living cells over other CPPs such as the HIV-1 Tat NLS [Vives, E. et al. (1997) J. Biol. Chem. 272, 16010-16017; Futaki, S. et al. (2000) J. Biol. Chem. 17] is that S4₁₃ is an “inert peptide” lacking any known intracellular function.

A further aspect of the invention aims to provide a pharmaceutical composition for intracellular delivery of a pharmaceutically active constituent, wherein said composition comprises as active ingredient a chimeric molecule as defined by the invention.

Thus, the pharmaceutical composition of the invention is intended for the delivery of a pharmaceutically active constituent into a non-nuclear or nuclear intracellular compartment.

The pharmaceutically active constituent to be delivered by the composition of the invention may be any one of, but not limited to, amino acids, oligopeptides, small peptides (up to 20 amino acids), polypeptides, proteins, nucleotides, oligonucleotides, nucleic acids and drugs. For the delivery of said pharmaceutically active constituent into the nuclear compartment, the composition of the invention should preferably comprise a chimeric molecule comprising a peptide with NLS-like properties.

The preparation of pharmaceutical compositions is well known in the art and has been described in many articles and textbooks, see e.g., Gennaro A. R. ed. (1990) Remington's Pharmaceutical Sciences, Mack Publishing Company, Easton, Pa., and especially pages 1521-1712 therein.

An additional aspect of the invention is the use of the chimeric molecule of the invention as a system for the delivery of an active constituent from an extracellular compartment into the intracellular compartment, wherein said intracellular compartment may be a non-nuclear or a nuclear intracellular compartment, and said active constituent is selected from the group consisting of amino acids, oligopeptides, small peptides, polypeptides, proteins, nucleotides, oligonucleotides, nucleic acids, drugs and fluorescently, radioactively or magnetically labeled chemical moieties.

The chimeric molecule to be used for the delivery of an active constituent from the extracellular into the nuclear compartment should preferably comprise a peptide having NLS-like properties.

As demonstrated by the inventors, fluorescently labeled dermaseptin-derived peptide S4₁₃ was able to penetrate into the cells and it accumulated in the cytoplasm. However, upon fusing the dermaseptin-derived peptide S4₁₃ with a peptide possessing NLS-like properties, as for example the SV40 T-Ag NLS or the ARM sequence from the Rev protein, the chimeric molecule was able to enter cell nuclei.

The chimeric molecule as defined in the invention is also intended to be used in the preparation of a pharmaceutical composition for the delivery of an active constituent from an extracellular compartment into the intracellular compartment.

As will become further clear in the following Examples, the invention is specially original for providing fusion peptides comprising a dermaseptin-derived peptide and a peptide having nuclear localization signal (NLS)-like properties.

In preferred embodiments, the dermaseptin-derived peptide comprises one of the sequences substantially as defined in SEQ ID NO:2, SEQ ID NO:3, SEQ ID NO:4 or SEQ ID NO:6, as well as functional analogues, derivatives or fragments thereof.

SEQ ID NO:2 is essentially a 13 amino acid peptide derived from Dermaseptin S4, also referred to as S4₁₃, as previously described [Mor, A., and Nicolas, P. (1994) id ibid.; Feder, R. et al. (2000) J. Biol. Chem. 275, 4230-4238]. SEQ ID NO:3 and SEQ ID NO:4 comprise the peptide S4₁₃ fused to the NLS sequence derived from the SV40 T-Ag, wherein the NLS sequence is positioned at the C-terminus or N-terminus, respectively. SEQ ID NO:6 is essentially the peptide S4₁₃ fused to the ARM sequence of the Rev protein, which has NLS-like properties.

The fusion peptide as defined in the invention is intended to be used as a delivery system for substances from the extracellular environment to an intracellular compartment.

Alternatively, the invention relates to the use of said fusion peptide as a delivery system for transport of substances from the extracellular vicinity to an intracellular compartment.

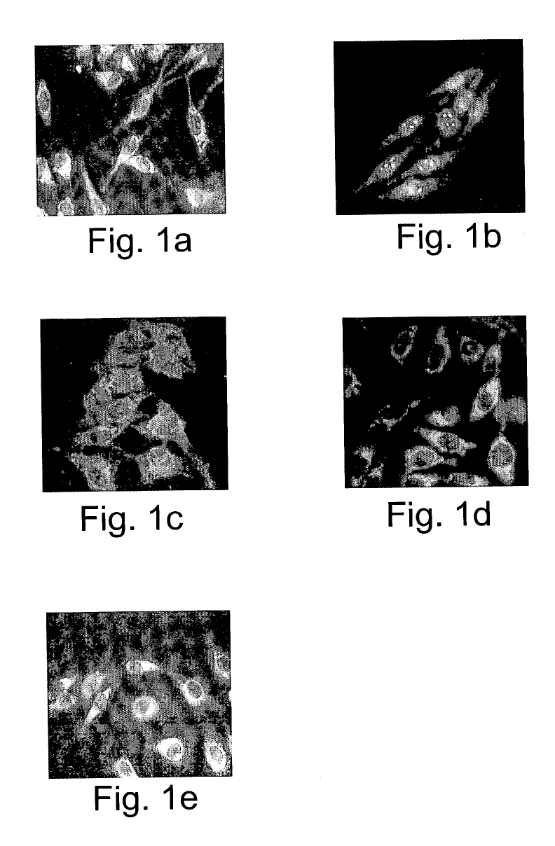

In sum, in the present work the inventors show that a peptide derived from Dermaseptin S4, S4₁₃ [Mor, A., and Nicolas, P. (1994) id ibid.; Feder, R. et al. (2000) id ibid.] efficiently penetrates plasma membranes of intact mammalian cultured cells. Being of low molecular weight and composed of only 13 amino acids, it was expected that such a small peptide would freely diffuse via the Nuclear Pore Complex (NPC) [Gorlich, D., and Mattaj, I. W. (1996) Science 271, 1513-1518; Gorlich, D. (1998) EMBO J. 17, 2721-2727] and accumulate within the intranuclear space of the recipient cells. However, surprisingly, the results show that the S4₁₃ peptide was non-karyophilic, and was retained within the cytoplasm without being translocated into the cells' nuclei. Nuclear import was then conferred upon S4₁₃ through the covalent attachment of peptides bearing an NLS, such as the SV40 T-Ag NLS or the Rev ARM [Malim, M. H. et al. (1989) Nature 338, 254-257]. Incubation of the S4₁₃-NLS fusion peptides with intact cultured HeLa cells resulted in their localization within the intranuclear space (shown in FIG. 1b). Nuclear import of this karyophilic peptide exhibited the same features that characterize active nuclear import.

Hence, the chimeric molecule of the invention or the pharmaceutical composition comprising the chimeric molecule of the invention may be used as a carrier or vector for gene therapy, or as a carrier for the intracellular transport of drugs or any other intracellular active molecules. Alternatively, the invention may be used, for example, as a diagnostic tool for magnetic resonance imaging (MRI).

In view of the inventors' findings, it is the intention of the present invention to additionally provide a method of screening for a cell-permeable nuclear import inhibitor, wherein said method comprises the following steps:

a. providing cells, contacting said cells with a chimeric molecule as defined in the invention;

b. contacting said cells with a candidate substance;

c. detecting the import of said chimeric molecule into the nuclei of said cells;

whereby the absence of import of the chimeric molecule indicates that said candidate substance is an inhibitor of nuclear import. In this method, the chimeric molecule should preferably comprise a peptide having NLS-like properties. Most preferably, the chimeric molecule should comprise a peptide essentially as defined in any one of SEQ ID NO:3, SEQ ID NO:4 or SEQ ID NO:6, as well as functional analogues, derivatives or fragments thereof.

Lastly, the invention provides a method for detecting changes in intracellular levels of proteins and nucleic acids, as well as of fragments thereof, comprising the following steps:

a. providing cells, contacting said cells with a chimeric molecule of the invention, wherein said chimeric molecule comprises a peptide having NLS-like properties, in which case said dermaseptin-derived peptide, together with said NLS-like peptide, preferably comprise the sequence substantially as defined in any one of SEQ ID NO:3, SEQ ID NO:4 or SEQ ID NO:6, as well as functional analogues, derivatives or fragments of any of said sequences, wherein at least one of said active constituents of the chimeric molecule of the invention can bind to the nucleic acid or protein whose levels are to be measured, and which chimeric molecule may optionally further comprise a second active constituent comprising a fluorescently, radioactively or magnetically labeled chemical moiety;

b. detecting the amount of said chimeric molecule in the cytoplasm and/or nuclei of said cells by suitable means; and

c. comparing the results obtained with an established control value of the non-nuclear and/or nuclear level of the nucleic acid or protein of interest, respectively;

whereby a level of the nucleic acid or protein of interest that is different from the control value of said nucleic acid or protein indicates a change on its level.

Thus, the invention may be used for identifying oscillations in gene expression levels due to certain pathological conditions.

Disclosed and described, it is to be understood that this invention is not limited to the particular examples, process steps, and materials disclosed herein as such process steps and materials may vary somewhat. It is also to be understood that the terminology used herein is used for the purpose of describing particular embodiments only and not intended to be limiting since the scope of the present invention will be limited only by the appended claims and equivalents thereof.

It must be noted that, as used in this specification and the appended claims, the singular forms “a”, “an” and “the” include plural referents unless the content clearly dictates otherwise.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The following Examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

### EXAMPLES

Experimental Procedures

Chemicals

Protected amino acids, Rink amide MBHA resin and coupling reagents were purchased from NOVA Biochem (Laufelfingen, Switzerland). Other chemicals were purchased from Sigma (St. Louis, USA) or Merck Darmstadt, Germany. Solvents for peptide synthesis were purchased from Baker, Phillipsburg, N.J., USA.

Cultured Cells

HeLa cell monolayers were grown in DMEM growth medium supplemented with 10% FCS, 0.3 gr/lit L-glutamine, 100 U/ml penicillin and 100 U/ml streptomycin (Beit Haemek, Israel). Cells were incubated at 37° C. in 5% CO₂ atmosphere and re-cultured every 4 days.

Peptide Synthesis-Fluorescent Labeling of the Synthetic Peptides

The peptides described in the present work were synthesized according to the SPPS method, using an Applied Biosystems Peptide synthesizer model 433A on Rink amide resin (loading 0.65 mmol/gr) by the standard Fmoc chemistry procedure.

The Fmoc protecting group was removed from the peptidyl-resin by treatment with 20% piperidine in DMF for 30 minutes. The peptides were labeled at the N-terminus as follows: Lissamine Rhodamine Sulfonyl Chloride (10 mg/ml; Molecular Probes) and DIEA (7 eq, 3.4 mmol) were dissolved in dry DMF and were added to the peptidyl resin. The reaction mixture was stirred in the dark for 24 hours. The peptidyl resin was washed with DMF×5 and with DCM×2. The peptides were de-protected and cleaved from the resin with trifluoroacetic acid (TFA) containing 1% anisole and 1% TDW as scavengers at 0° C. for 30 minutes and at room temperature for another 2 hours. The TFA was evaporated under nitrogen and the peptides were precipitated by cold ether, washed three times with ether, dissolved in TDW and lyophilized. Following purification by reversed phase HPLC using C-18 column (Acetonitrile/TDW containing 0.1% TFA wavelength 220 nm and 600 nm) the resulted peptides were characterized by TOF-MS.

TABLE 1

Amino acid sequence
of the various peptides used.

| Peptide | | Sequence |
|---|---|---|
| Name | Peptide Sequence^{a} | ID |
| Derma- | ALWMTLLKKVLKAAAKAALNAVLVGANA- | SEQ ID NO:1 |
| septin- | COOH | |
| S4 | | |
| S4₁₃ | ALWKTLLKKVLKA-NH₂ | SEQ ID NO:2 |
| S4₁₃-PV | ALWKTLLKKVLKAPKKKRKV-NH₂ | SEQ ID NO:3 |
| PV-S4₁₃ | PKKKRKVALWKTLLKKVLKA-NH₂ | SEQ ID NO:4 |
| rPV-S4₁₃ | VKRKKKPALWKTLLKKVLKA-NH₂ | SEQ ID NO:5 |
| RR-S4₁₃ | RQARRNRRRALWKTLLKKVLKA-NH₂ | SEQ ID NO:6 |
| Rev ARM | RQARRNRRRC-NH₂ | SEQ ID NO:7 |
| Tat ARM | GRKKRRQRRRPPQC-NH₂ | SEQ ID NO:8 |

^{a}The S4₁₃ sequence is underlined.

Penetration of the Synthetic Peptides into Intact Cultured Cells: Microscopic Observations

HeLa cells (3×10⁴ per well) were cultured on eight-well Lab-Teck cover slips (Nunc Inc.) or on 10-mm cover slips to sub confluent density. Following the removal of the culture medium, the cells were washed three times with PBS and then exposed to different concentrations of the Lissamine Rhodamine-labeled peptides at 37° C. or at 4° C. At the end of the incubation period the cells were washed three times in PBS and fixed in 4% (v/v) formaldehyde dissolved in PBS. Fixed cells were examined by fluorescence microscopy (Zeiss Germany, a 40× objective; Apoplan) or by confocal microscopy using an MRC 1024 confocal imaging system (Bio-Rad). The microscope (Axiovert 135M; Zeiss Germany, a 63× objective; Apoplan; NA 1.4) was equipped with an argon ion laser for Rhodamine excitation at 514 nm (emission 580).

Determination of Nuclear Uptake in Permeabilized Cells—Fluorescent Microscopy Observations

HeLa cells were cultivated on 10 mm cover slips to sub confluent density and then permeabilized with digitonin as described previously [Friedler, A. et al. (1998) Biochemistry 37, 5616-5622]. The cells were incubated with the peptides and nuclear import was followed by fluorescence microscopy as described [Friedler, A. et al. (1998) id ibid.].

The Effect of the Various Peptides on Cell Viability

To study the effect of the peptides on cell viability, increasing concentrations (1-15 μM) of the peptides were added to cultured cells (96 well 3×10⁴ cells per well in DMEM). Following incubation at 37° C. for 30 minutes 100 ml, Trypan Blue solution was added (0.4% in HBSS buffer; Sigma) and HBSS buffer (5:3) and viable cells counted after 5 minutes of continuous stirring. Cell death was not greater than 20%.

Microinjection of the Peptides into HeLa Cells

HeLa cells were microinjected with either S4₁₃ or with RR-S4₁₃ using the CompInject AIS2 automated microinjection system (Cell Biology Trading, Hamburg, Germany) as described previously [Neumann, M. et al. (2001) J. Cell Sci. 114, 1717-29] using a microinjection method developed by Graessmann et al., as described [Graessmann, M. and Graessmann, A. (1983) Methods Enzymol. 101, 482-492].

### Example 1

### S4₁₃ is a Non-Karyophilic Peptide

The results shown in FIG. 1a demonstrate that fluorescently labeled S4₁₃, a 13 amino acid peptide (Table 1) derived from the antimicrobial peptide Dermaseptin S4, readily penetrated intact cultured HeLa cells. The S4₁₃ peptide essentially accumulated within the cells' cytoplasm and not in the cells' nuclei despite its low molecular weight. The same was observed for its parent peptide, S4 (data not shown). Penetration of S4₁₃ into intact cells occurred at 37° C., as well as at 4° C. (FIG. 1d), and even in ATP depleted cells, indicating a non-metabolic process. Kinetic studies revealed that penetration of S4₁₃ into cultured HeLa cells was relatively fast, occurring within 5 minutes of incubation with 1 μM of the peptide either at 37° C. or at 4° C. S4₁₃ retained its non-karyophilic properties and remained in the cell cytoplasm with very little, if any, nuclear localization even 24 hours after incubation at 37° C. (not shown). Highly clustered intracellular fluorescent dots were observed in intact HeLa cells incubated with Lissamine Rhodamine (LR)-labeled S4₁₃. This may indicate a process of self-aggregation that results in the formation of aggregates, which may be too large for passive diffusion into the nuclei.

Interestingly, peptide S4₁₃ promoted the co-penetration of small molecules into intact cells such as the fluorophore LR. Incubation of HeLa cells with a mixture of unlabeled S4₁₃ and free LR resulted in the appearance of a few intracellular fluorescent dots, which did not occur in cells incubated with the LR alone (Table 2). S4₁₃ was non-toxic at the concentrations used as was indicated by the Trypan Blue test (not shown, see Experimental Procedures).

TABLE 2
Cell penetration and nuclear import of various dermaseptin-
derived peptides.

| | |
|---|---|
| Incubation | Penetration |

| Peptides | Temperature | Cytopl. | Nucl. |
|---|---|---|---|
| S4 | 37° C. | + | − |
| S4₁₃ | 37° C. | + | − |
| PV-S4₁₃ | 37° C. | + | + |
| S4₁₃-PV | 37° C. | + | + |
| rPV-S4₁₃ | 37° C. | + | − |
| S4₁₃ | 4° C. | + | − |
| PV-S4₁₃ | 4° C. | + | − |
| PV-S4₁₃ | 37° C. | + | − |
| ATP-depleted system^{a} | | | |
| S4₁₃ + unlabeled PV-S4₁₃ | 37° C. | + | − |
| (1:100) | | | |
| PV-S4₁₃ + unlabeled PV- | 37° C. | + | − |
| S4₁₃(1:100) | | | |
| L-Rhodamine | 37° C. | − | − |
| S4₁₃ + L-Rhodamine | 37° C. | + | − |
| PV-S4₁₃ + L-Rhodamine | 37° C. | + | − |

^{a}Cells were pre-incubated with 0.25 mM 2, 4 dinitrophenol for 30 minutes prior to the addition of the peptides.
Abbreviations:
Cytop., cytoplasm;
Nucl., nucleus.

Labeled peptides were incubated for 5 minutes (1 mM) at the indicated temperature with a monolayer of cultured HeLa cells. Following fixation, the various samples were examined by fluorescence microscopy.

### Example 2

### The NLS of SV40 T-Antigen Confers Karyophilic Properties Upon S4₁₃

Composite peptides bearing both the sequences of the S4₁₃ peptide as well as the NLS motif of the SV40-T-antigen were obtained by the synthesis of fused peptides containing both sequences (Table 1 and Experimental Procedures). This was done in order to find out whether the addition of an NLS could confer karyophilic properties upon S4₁₃ without affecting its cell permeability properties. The SV40-T-antigen-NLS was covalently attached either to the N- or to the C-terminus of S4₁₃ resulting in composite peptides designated PV-S4₁₃ and S4₁₃-PV, respectively. Also, the reverse sequence of the SV40-T-antigen-NLS was attached to S4₁₃ yielding the peptide rPV-S4₁₃ (Table 1).

The results shown in FIG. 1 and in Table 2 show that incubation of PV-S4₁₃ (FIG. 1b) and S4₁₃-PV (not shown) with intact cultured HeLa cells resulted in cell penetration and accumulation within the cells' nuclei. Evidently, both peptides were cell permeable like S4₁₃ but unlike S4₁₃, the PV-S4₁₃ and S4₁₃-PV peptides possessed karyophilic properties. NLS-mediated nuclear import is suggested by the results showing that the peptide rPV-S4₁₃, which is a S4₁₃ conjugate with a non-functional NLS, was much less karyophilic then the PV-S4₁₃ and showed very little, if any, accumulation within the cells' nuclei/nucleoli (FIG. 1c and Table 2).

Similar to S4₁₃, the composite PV-S4₁₃ and S4₁₃-PV peptides penetrated intact HeLa cells at 37° C. as well as at 4° C. (FIG. 1). However at 4° C. the PV-S4₁₃ and S4₁₃-PV peptides retained in the cytoplasm, strengthening the view that the nuclear import observed at 37° C. was an active process. Cell viability tests revealed that like S4₁₃, PV-S4₁₃ and S4₁₃-PV peptides were not toxic at the concentrations used (not shown).

### Example 3

### Import of PV-S4₁₃ and S4₁₃-PV into Nuclei of Permeabilized HeLa Cells

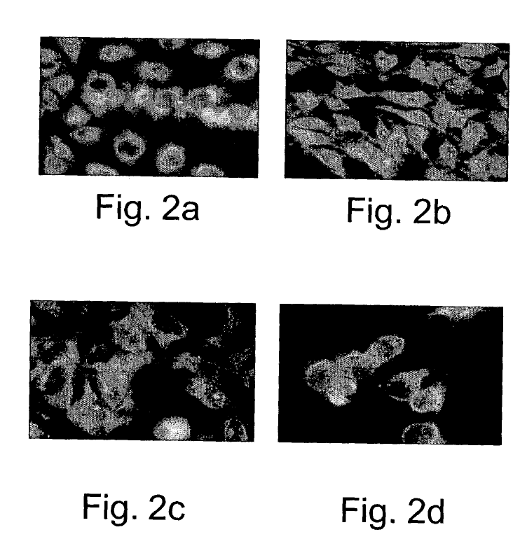

The results in FIG. 2 and in Table 3 show that the peptide S4₁₃ did not penetrate even nuclei of digitonin permeabilized HeLa cells. On the other hand, import of PV-S4₁₃ into nuclei of permeabilized HeLa cells was absolutely dependent on the addition of a reticulocyte extract (FIGS. 2b and d), indicating that its translocation had the same features that characterize SV40 T-Ag-NLS-mediated nuclear import [Gorlich, D. (1997) Curr. Opin. Cell. Biol. 9, 412-419; Broder, Y. C. et al. (1997) FEBS Lett. 412, 535-539], namely required exogenously added cytosolic factors. In contrast, the peptide rPV-S4₁₃ either did not accumulate or showed poor nuclei accumulation both in the presence and in the absence of the cytosolic extract. This indicates that the nuclear import of PV-S4₁₃ and S4₁₃-PV was receptor-dependent and mediated by the attached NLS. Nuclear import of PV-S4₁₃ and S4₁₃-PV was ATP-dependent, inhibited by GTPγS, WGA and excess of free unlabeled SV40 T-Ag-NLS peptide, and did not occur at 4° C. (Table 3). Interestingly, it was also inhibited by excess unlabeled PV-S4₁₃ but not by S4₁₃ itself. All these results clearly show that nuclear import of PV-S4₁₃ is characterized by the same features that characterize active import of karyophilic proteins. Identical results to those observed with PV-S4₁₃ were obtained when S4₁₃-PV (S4₁₃ bearing an NLS sequence at its C terminus), was used as a transport substrate (not shown).

TABLE 3
Requirement and characterization of PV-S4₁₃ peptide nuclear
import in permeabilized HeLa cells.

| Experimental | | Nuclear Import of |
|---|---|---|
| conditions | | PV-S4₁₃ |
| (i) | Complete System | + |
| (ii) | Hexokinase (ATP depleted system) | − |
| (iii) | As (i), in the absence of reticulocyte | − |
| | extract | |
| (iv) | As (i) at 4° C. | − |
| (v) | As (i) + GTPγS | − |
| (vi) | As (i) + SV40-NLS | +/− |
| (vii) | As (i) + S4₁₃ | + |
| (viii) | As (i) + PV-S4₁₃ | +/− |

+: Most of the nuclei in the microscopic fields are highly fluorescent;
−: No fluorescence in the nuclei;
+/−: Most of the nuclei are very weakly fluorescent.

Experimental conditions of nuclear import in digitonin-permeabilized cells (complete system) were as described. PV-S4₁₃ was used at 1 mM and all incubations were performed at 37° C. in the presence of reticulocyte lysate, unless otherwise indicated. Additions: GTPγS (20 mM) and the following unlabeled peptides SV40 T-Ag NLS, S4₁₃ and PV-S4₁₃ at a ratio of 1:100 (mol/mol).

### Example 4

### Rev-ARM-Mediated Nuclear Import of S4₁₃ (RR-S4₁₃) in Intact and Permeabilized HeLa Cells

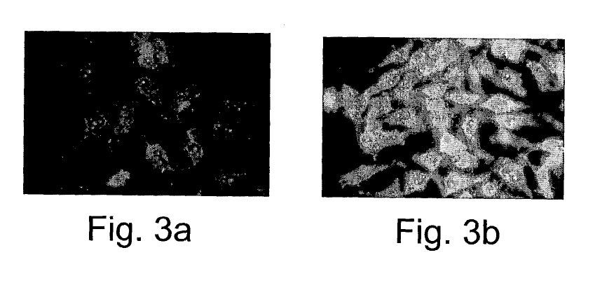

The results in FIG. 3 and in Table 4 show that a peptide bearing the Rev ARM, namely RR-S4₁₃ (see Table 1 and Experimental Procedures) readily penetrated into intact HeLa cells and accumulated—within a short period of time—within the nuclei of these cells. Nuclear import was also observed when RR-S4₁₃ was incubated with digitonin permeabilized HeLa cells. However, nuclear import of RR-S4₁₃ did not require the addition of cytosolic extract [Friedler, A. et al. (1999) J. Mol. Biol. 289, 431-437]. Active and specific nuclear import is evident from the results showing that in intact cells, as well as in permeabilized cells, nuclear import was ATP dependent and inhibited by excess unlabeled RR-S4₁₃ as well as by other ARM-peptides such as those bearing the Rev and Tat ARM (Tables 4 and 5). The inhibition observed by the externally added ARM peptides clearly indicated that these peptides are cell permeable. This indeed can be inferred from results in FIG. 3, which demonstrate that the Rev-ARM, similar to what has been demonstrated for Tat ARM peptide [Vives, E. et al. (1997) id ibid.], is cell permeable [Futaki et al. (2000) id ibid.]. Excess unlabeled RR-S4₁₃ also blocked nuclear import of a Rev-ARM-BSA conjugate into the nuclei of permeabilized cells as well as of RR-S4₁₃ in intact cells. Such inhibition was not observed by the SV40 T-Ag-NLS (Tables 4 and 5).

TABLE 4
Cell penetration and nuclear import of the ARM synthetic peptides

| | | Incubation | Penetration | |
|---|---|---|---|---|
| Peptides | | Temp. | Cytop. | Nuclei |
| (i) | Tat-ARM^{a} | 37° C. | + | + |
| (ii) | Rev-ARM^{b} | 37° C. | + | + |
| (iii) | RR-S4₁₃^{c} | 37° C. | + | + |
| (iv) | Tat-ARM | 4° C. | + | + |
| (v) | Rev-ARM | 4° C. | + | + |
| (vi) | RR-S4₁₃ | 4° C. | + | − |
| (vii) | As (vi) + unlabeled RR-S4₁₃ | 37° C. | + | − |
| (viii) | As (vi) + unlabeled Rev-ARM | 37° C. | + | − |
| (ix) | As (vi) + unlabeled Tat-ARM | 37° C. | + | − |

^{a}1 mM,
^{b}10 mM,
^{c}2 mM.
Abbreviations:
Temp., temperature;
Cytop., cytoplasm.

Synthetic peptides bearing Tat and Rev ARM sequences were synthesized and fluorescently labeled as described. Molar ratio between unlabeled peptides and RR-S4₁₃ was always 1:400 (mol/mol).

TABLE 5
Nuclear import of RR-S4₁₃ into the nuclei of permeabilized HeLa
cells.

| Experimental conditions | | Nuclear uptake |
|---|---|---|
| (i) | In the absence of cytosolic factors | + |
| (ii) | In the presence of cytosolic factors | +/− |
| (iii) | ATP depleted cells | − |
| (iv) | Incubation in 4° C. | − |
| (v) | In the presence of unlabeled RR-S4₁₃ | +/− |
| (vi) | In the presence of unlabeled Rev-ARM | − |
| (vii) | In the presence of unlabeled Tat-ARM | +/− |
| (viii) | In the presence of unlabeled SV40 T-Ag NLS | + |
| (ix) | LR-BSA-Rev + unlabeled RR-S4₁₃ | − |

+: Most of the nuclei in the microscopic fields are highly fluorescent;
−: No fluorescence in the nuclei;
+/−: Most of the nuclei are very weakly fluorescent.

Unlabeled peptides RR-S4₁₃, SV40 T-Ag-NLS, Rev and Tat ARM were added at a peptide ratio of 1:100 (mole/mole). Determination of cell viability (data not shown) revealed that also the ARM derived peptides Tat, Rev and RR-S4₁₃ were not toxic to the cells at concentrations up to about 15 mM.

The results in FIG. 4 show that following microinjection into intact cells, the peptides S4₁₃ retained in the cytoplasm while RR-S4₁₃ accumulated within the intranuclear space of these cells. Once again, these results prove the non-karyophilic and karyophilic properties of S4₁₃ and RR-S4₁₃, respectively.

1. A chimeric molecule comprising a dermaseptin-derived peptide and at least one biologically or pharmaceutically active constituent.

2. A chimeric molecule comprising: a dermaseptin-derived peptide, a peptide having nuclear localization signal (NLS)-like properties, and at least one biologically or pharmaceutically active constituent.

3. The chimeric molecule of claim 1, wherein said dermaseptin-derived peptide comprises the sequence substantially as defined in SEQ ID NO:2 or functional analogues, derivatives or fragments thereof, and wherein said active constituent is selected from the group consisting of amino acids, oligopeptides, small peptides, polypeptides, proteins, nucleotides, oligonucleotides, nucleic acids, drugs and fluorescently, radioactively or magnetically labeled chemical moieties.

4. The chimeric molecule of claim 2, wherein said dermaseptin-derived peptide, together with the peptide having NLS-like properties, comprise the sequence substantially as defined in any one of SEQ ID NO:3, SEQ ID NO:4 and SEQ ID NO:6, and functional analogues, derivatives or fragments of any of said sequences, and wherein said active constituent is selected from the group consisting of amino acids, oligopeptides, small peptides, polypeptides, proteins, nucleotides, oligonucleotides, nucleic acids, drugs and fluorescently, radioactively or magnetically labeled chemical moieties.

5. A composition for intracellular delivery of an active constituent, comprising as active ingredient a chimeric molecule as defined in claim 1, optionally further comprising a physiologically acceptable diluent, carrier, additive and/or excipient, and wherein said active constituent is selected from the group consisting of amino acids, oligopeptides, small peptides, polypeptides, proteins, nucleotides, oligonucleotides, nucleic acids and drugs.

6. A composition for intracellular delivery of an active constituent, comprising as active ingredient a chimeric molecule as defined in claim 2, optionally further comprising a physiologically acceptable diluent, carrier, additive and/or excipient, and wherein said active constituent is selected from the group consisting of amino acids, oligopeptides, small peptides, polypeptides, proteins, nucleotides, oligonucleotides, nucleic acids and drugs.

7. A composition for intracellular delivery of an active constituent, comprising as active ingredient a chimeric molecule as defined in claim 3, optionally further comprising a physiologically acceptable diluent, carrier, additive and/or excipient, and wherein said active constituent is selected from the group consisting of amino acids, oligopeptides, small peptides, polypeptides, proteins, nucleotides, oligonucleotides, nucleic acids and drugs.

8. A composition for intracellular delivery of an active constituent, comprising as active ingredient a chimeric molecule as defined in claim 4, optionally further comprising a physiologically acceptable diluent, carrier, additive and/or excipient, and wherein said active constituent is selected from the group consisting of amino acids, oligopeptides, small peptides, polypeptides, proteins, nucleotides, oligonucleotides, nucleic acids and drugs.

9. A composition for intracellular delivery of an active constituent, comprising as active ingredient a chimeric molecule as defined in claim 3, for the delivery of an active constituent into a non-nuclear intracellular compartment, optionally further comprising a physiologically acceptable diluent, carrier, additive and/or excipient, and wherein said active constituent is selected from the group consisting of amino acids, oligopeptides, small peptides, polypeptides, proteins, nucleotides, oligonucleotides, nucleic acids and drugs.

10. A composition for intracellular delivery of an active constituent, comprising as active ingredient a chimeric molecule as defined in claim 4, for the delivery of an active constituent into an intranuclear cellular compartment, optionally further comprising a physiologically acceptable diluent, carrier, additive and/or excipient, and wherein said active constituent is selected from the group consisting of amino acids, oligopeptides, small peptides, polypeptides, proteins, nucleotides, oligonucleotides, nucleic acids and drugs.

11. A chimeric molecule as defined in claim 1, for use as a system for the delivery of at least one active constituent from an extracellular compartment into the intracellular compartment, wherein said active constituent is selected from the group consisting of amino acids, oligopeptides, small peptides, polypeptides, proteins, nucleotides, oligonucleotides, nucleic acids, drugs and fluorescently, radioactively or magnetically labeled chemical moieties.

12. A chimeric molecule as defined in claim 2, for use as a system for the delivery of at least one active constituent from an extracellular compartment into the intracellular compartment, wherein said active constituent is selected from the group consisting of amino acids, oligopeptides, small peptides, polypeptides, proteins, nucleotides, oligonucleotides, nucleic acids, drugs and fluorescently, radioactively or magnetically labeled chemical moieties.

13. A chimeric molecule as defined in claim 3, for use as a system for the delivery of at least one active constituent from an extracellular compartment into the intracellular compartment, wherein said active constituent is selected from the group consisting of amino acids, oligopeptides, small peptides, polypeptides, proteins, nucleotides, oligonucleotides, nucleic acids, drugs and fluorescently, radioactively or magnetically labeled chemical moieties.

14. A chimeric molecule as defined in claim 4, for use as a system for the delivery of at least one active constituent from an extracellular compartment into the intracellular compartment, wherein said active constituent is selected from the group consisting of amino acids, oligopeptides, small peptides, polypeptides, proteins, nucleotides, oligonucleotides, nucleic acids, drugs and fluorescently, radioactively or magnetically labeled chemical moieties.

15. The molecule of claim 11, wherein said intracellular compartment is a non-nuclear intracellular compartment.

16. The molecule of claim 13, wherein said intracellular compartment is a non-nuclear intracellular compartment.

17. The molecule of claim 12, wherein said intracellular compartment is a nuclear compartment.

18. The molecule of claim 14, wherein said intracellular compartment is a nuclear compartment.

19. Use of a chimeric molecule as defined in claim 3, in the preparation of a composition for the delivery of an active constituent from an extracellular compartment into the intracellular compartment, comprising the step of admixing the chimeric molecule as defined in claim 3 with a physiologically acceptable diluent, carrier, additive and/or excipient.

20. Use of a chimeric molecule as defined in claim 4, in the preparation of a composition for the delivery of an active constituent from an extracellular compartment into the intracellular compartment, comprising the step of admixing the chimeric molecule as defined in claim 4 with a physiologically acceptable diluent, carrier, additive and/or excipient.

21. A fusion peptide comprising a dermaseptin-derived peptide and a peptide having nuclear localization signal (NLS)-like properties.

22. The fusion peptide of claim 21, wherein said dermaseptin-derived peptide comprises the sequence substantially as defined in SEQ ID NO:2.

23. The fusion peptide of claim 21, wherein the sequence of said fusion peptide is selected from any one of SEQ ID NO:3, SEQ ID NO:4 and SEQ ID NO:6.

24. The fusion peptide of claim 23, for use as a delivery system for substances from the extracellular to the intracellular compartment.

25. The fusion peptide of claim 23, for use as a delivery system for substances from the extracellular to the nuclear cellular compartment.

26. A fusion peptide as defined in claim 22, for use as a delivery system for transport of substances from the extracellular to the intracellular compartment.

27. A fusion peptide as defined in claim 23, for use as a delivery system for transport of substances from the extracellular to the intracellular compartment.

28. A method of screening for a cell-permeable nuclear import inhibitor comprising the following steps:
a. providing cells, contacting said cells with a chimeric molecule as defined in claim 4;
b. contacting said cells with a candidate substance;
c. detecting the import of said chimeric molecule into the nuclei of said cells;
whereby the absence of import of the chimeric molecule indicates that said candidate substance is an inhibitor of nuclear import.

29. A method for detecting changes in intracellular levels of proteins and nucleic acids, as well as of fragments thereof, comprising the following steps:
a. providing cells, contacting said cells with a chimeric molecule as defined in claim 4, wherein at least one of said active constituent of the chimeric molecule is able to bind to the nucleic acid or protein whose levels will be measured, and optionally comprising a second active constituent comprised of a fluorescently, radioactively or magnetically labeled chemical moiety;
b. detecting the amount of said chimeric molecule in the cytoplasm and/or nuclei of said cells by suitable means; and
c. comparing the results obtained with an established control value of the non-nuclear and/or nuclear level of the nucleic acid or protein of interest, respectively.


SEQUENCE LISTING (8 sequences)

SEQ ID NO: 1 - protein, 28 aa, Artificial Sequence
  Description of Artificial Sequence Dermaseptin S4
ALWMTLLKKVLKAAAKAALNAVLVGANA

SEQ ID NO: 2 - protein, 13 aa, Artificial Sequence
  Description of Artificial Sequence Dermaseptin-derived peptide S4-13
ALWKTLLKKVLKA

SEQ ID NO: 3 - protein, 20 aa, Artificial Sequence
  Description of Artificial Sequence fusion peptide S4-13 and SV40 NLS at C-terminus
ALWKTLLKKVLKAPKKKRKV

SEQ ID NO: 4 - protein, 20 aa, Artificial Sequence
  Description of Artificial Sequence fusion peptide S4-13 and SV40 NLS at N-terminus
PKKKRKVALWKTLLKKVLKA

SEQ ID NO: 5 - protein, 20 aa, Artificial Sequence
  Description of Artificial Sequencefusion peptide S4-13 and reverse SV40 NLS
VKRKKKPALWKTLLKKVLKA

SEQ ID NO: 6 - protein, 22 aa, Artificial Sequence
  Description of Artificial Sequence fusion peptide S4-13 and ARM
RQARRNRRRALWKTLLKKVLKA

SEQ ID NO: 7 - protein, 10 aa, Artificial Sequence
  Description of Artificial Sequence Rev ARM
RQARRNRRRC

SEQ ID NO: 8 - protein, 14 aa, Artificial Sequence
  Description of Artificial Sequence Tat ARM
GRKKRRQRRRPPQC